(19) United States
(12) Patent Application Publication
(10) Pub. No.: US 2015/0257718 A1
(43) Pub. Date: Sep. 17, 2015

(54) REALTIME IMAGING AND RADIOTHERAPY OF MICROSCOPIC DISEASE

(71) Applicant: THE REGENTS OF THE UNIVERSITY OF CALIFORNIA, Oakland, CA (US)

(72) Inventors: Keisuke S. Iwamoto, Los Angeles, CA (US); Magnus Dahlbom, Tarzana, CA (US); John J. Demarco, Torrance, CA (US); Dan Ruan, Los Angeles, CA (US)

(73) Assignee: The Regents of the University of California, Oakland, CA (US)

(21) Appl. No.: 14/431,716

(22) PCT Filed: Sep. 27, 2013
(86) PCT No.: PCT/US13/62480

§ 371 Date: Mar. 26, 2015

Related U.S. Application Data
(60) Provisional application No. 61/707,879, filed on Sep. 28, 2012.

(51) Int. Cl. A61B 6/03; A61N 5/10; A61M 31/00; A61B 6/00

(52) U.S. Cl. CPC A61B 6/037; A61B 6/4057; A61B 6/4258; A61B 6/4266; A61B 6/4452; A61B 6/4488; A61B 6/5217; A61N 5/1001; A61M 31/005; A61N 2005/1098

(57) ABSTRACT

Disclosed herein are methods, systems and therapeutics concerning radiotherapy of microscopic disease based on realtime imaging. The system comprises an intra-corporeal component and an extra-corporeal component. The intra-corporeal component comprises a detector/imaging subunit and a treatment subunit, where both subunits are placed in a cavity of a patient. The extra-corporeal component comprises a detector that is placed outside any cavity of the patient. Through this system, a treatment can be applied to a target tissue in a patient concurrently or within a short period time to signal detections.

### CROSS REFERENCE TO RELATED APPLICATION

The application claims priority to U.S. provisional application No. 61/707,879 filed on Sep. 28, 2012 and also entitled “Realtime Imaging and Radiotherapy of Microscopic disease,” which is hereby incorporated by reference herein in its entirety.

### FIELD OF THE INVENTION

The invention disclosed herein generally relates to methods, systems and therapeutics concerning radiotherapy of microscopic disease based on realtime imaging.

### BACKGROUND

Devices and methods for imaging sub-millimeter-sized tumors that are embedded in tissues (e.g., at depths greater than 1-2 mm) are not available. Consequently, methods for treating such tumors are also lacking due to the inability in combining high specific and sensitive imaging with highly conformal radiation.

What is needed are systems, methods and therapeutics that can overcome the deficiencies in the art.

### SUMMARY OF THE INVENTION

In oncology, the sequence of locating the tumor and then treating it is a sine qua non. The smaller the tumor is, the easier it is to treat, and the better is the prognosis. On the other hand, the smaller the tumor is, the more difficult it is to detect and target for treatment. Additionally, the smaller the tumor is, the more normal tissue is at risk from collateral damage especially if treatment accuracy is compromised by imprecise targeting. And finally, the greater the time interval and the number of procedural steps between localization and treatment, the greater is the risk of missing the target.

Provided herein are methods, systems and therapeutics concerning radiotherapy of microscopic disease based on realtime imaging. In one aspect, the system and method are based on High-Resolution Asymmetrically Positioned Positron Emission Tomography (HRAPPET) guided Intensity Modulated Radiation Therapy (IMRT), collectively as HRAPPET-IMRT. For example, in some embodiments, after administration of a radiotracer into the patient, an HRAPPET-IMRT system is used as an intraoperative device to simultaneously image sub-millimeter remnants of a tumor following surgical resection and to precisely treat them with x-irradiation from a miniature x-ray tube coupled to the detector, such as for brain or breast cancers.

Similarly, it can also be used as an intracavitary or intraluminal device to detect and treat small, early-stage tumors, such as endometrial cancers. Moreover, because of its unique imaging capabilities, it can be used as a screening method for early-stage tumors if radiation treatment is not indicated, as might be the case in ovarian cancers.

In one aspect, provided herein is a tomographic imaging system for realtime imaging of sub-millimeter-sized tumor clusters in a patient. The system comprises an intra-corporeal component and an extra-corporeal component.

In some embodiments, the intra-corporeal component comprises i) a first detector in a cavity within the body of a patient; and ii) a radiation source capable of emitting a therapeutic radiation, where said radiation source is also in the cavity within the body of a patient;

In some embodiments, the extra-corporeal component comprises a second detector outside any cavity of the body of the patient, where the second detector is configured to always face the imaging hemisphere of said first detector.

In some embodiments, a contrast agent, which modulates the intensity of the therapeutic radiation, wherein the first detector can be rotated with respect to the body of said patient.

In some embodiments, the contrast agent is selected from the group consisting of an iodinated agent, a high-Z material liquid, and a combination thereof.

In some embodiments, the first detector is a gamma detector.

In some embodiments, the second detector is a gamma detector.

In some embodiments, the radiation source is a miniature x-ray source. In some embodiments, the system further comprises another radiation source for imaging; for example, source of positron-emitters. A positron-emitting isotope labeled compound is administered to the patient.

In some embodiments, the radiation source further comprises an inlet and an outlet for an x-ray source coolant.

In some embodiments, wherein the radiation source further comprises an anode voltage lead and a cathode voltage lead.

In some embodiments, the radiation source further comprises an inlet and an outlet for the contrast agent.

In some embodiments, the first detector is placed in a cavity selected from the group consisting of the cranial cavity, cervical cavity, dorsal cavity, a spinal cavity, pelvic cavity, thoracic cavity, abdominal cavity and abdominopelvic cavity, ovarian cavity, gloiblastoma multiforme margin cavity, uterus, and a surgically created cavity.

In some embodiments, the radiation source can be rotated with respect to the body of said patient.

In some embodiments, the second detector can be rotated with respect to the body of said patient.

In some embodiments, the intra-corporeal component further comprises a stationary outer shell that does not rotate with the first detector.

In some embodiments, the intra-corporeal component further comprises a shield surrounding the radiation source. In some embodiments, the shield is removable.

In one aspect, provided herein is a method for imaging a sub-millimeter-sized tumor in a patient, comprising the steps of:

administering, to a patient in need, a contrast agent that is preferentially taken up by tumor cells;

placing an intra-corporeal component in a cavity within the body of a patient, wherein intra-corporeal component comprising:

i) a first detector; and ii) a radiation source capable of emitting a therapeutic radiation;

placing a second detector outside any cavity of the body of the patient, wherein the second detector is configured to always face the imaging hemisphere of said first detector;

collecting a first image of a first portion of said cavity using the first detector by using therapeutic x-rays whose intensity is modulated by said contrast agent;

rotating the first and second detector over a first angle while maintaining the relative configuration between them;

collecting a second image of a second portion of said cavity using the first detector, wherein the first and second portions do not overlap completely.

In some embodiments, the method further comprises a step of constructing a new image based on said first and second images.

In some embodiments, the contrast agent is selected from the group consisting of an iodinated agent, a high-Z material liquid, and a combination thereof. In some embodiments, the new image is a 3-dimensional image.

In some embodiments, the first detector is rotated at a first angle between 1 and 180 degrees. For example, the first angle can be between 1 and 30 degrees, 1 and 45 degrees, 1 and 60 degrees, 1 and 75 degrees, 1 and 90 degrees, 1 and 120 degrees, 1 and 150 degrees. In some embodiments, the first angle is about, 30, 45, 60, 75, 90, 120, 150, or 180 degrees.

### BRIEF DESCRIPTION OF THE DRAWINGS

Those of skill in the art will understand that the drawings, described below, are for illustrative purposes only. The drawings are not intended to limit the scope of the present teachings in any way.

FIGS. 1A and 1B illustrate exemplary configurations of the system.

FIG. 2 illustrates an exemplary use.

FIG. 3 illustrates an exemplary use.

FIG. 4 illustrates an exemplary use.

FIG. 5 illustrates an exemplary intra-corporeal component.

FIG. 6 illustrates exemplary intra-corporeal component parts in detail.

FIG. 7 illustrates exemplary intra-corporeal component parts configuration.

FIG. 8 illustrates an exemplary operation sequence.

FIGS. 9A through 9D illustrate an exemplary characterization of a source. 9A) X-ray spectra in air or water. 9B) Relative depth dose in water for two beam energies and with or without bolus as calculated by Monte Carlo simulation. 9C) Two-dimensional representation of B′. 9D) Clonogenic survival of U87MG human GBM cells and MCF7 human breast cancer cells for derivation of RBE compared to a cobalt-60 irradiator.

FIG. 10 illustrates an exemplary Ambi-cranial PET system with intracranial probe detector array and extracranial arc detector array.

FIG. 11 illustrates exemplary spatially-varying reconstruction properties.

FIGS. 12A and 12B illustrate exemplary Spatial varying point-spread-function from reconstruction: (A) Reconstruction behavior for pt source at (54,20) mm; (B) Reconstruction behavior for pt source at (164,20) mm.

### DETAILED DESCRIPTION OF THE INVENTION

Unless otherwise noted, terms are to be understood according to conventional usage by those of ordinary skill in the relevant art.

Currently, there are no methods: 1) to image sub-millimeter-sized tumors at tissue depths greater than 1-2 mm, 2) to treat such tumors, which lead to recurrences, without damaging large areas of normal tissues, and 3) that combine high specific and sensitive imaging with highly conformal radiation treatment of near-microscopic tumor clusters ‘hidden’ in a relatively larger volume of normal tissue. The systems and methods disclosed herein fulfill the need.

With respect to point #1: State-of-the-Art beta-probe imaging cannot resolve tumors on the order of a few millimeters at tissue depths over 1-2 mm. For deeper tumors, the limit of resolution with gamma-probes or even with State-of-the-Art conventional PET is well over 5 mm. Ultrasonic imaging devices are also utilized for deeper imaging, but specificity and sensitivity are unacceptably too low to locate and/or confirm malignancy of tumors smaller than 10 mm. Therefore, failure of detecting early stage cancers can result in late diagnosis and treatment in certain organs as is the case with ovarian cancer.

With respect to point #2: there are commercially available miniature x-ray tubes that are used as interstitial or intracavitary devices to irradiate tumors or tumor remnants without live tumor imaging/detection, thus relying on previously procured data. The irradiation volume of such electronic brachytherapy devices (miniature x-ray sources) are not conformed to the irregular deposition patterns of the tumor cells—leading to unnecessary irradiation of normal tissue. Current State-of-the-Art radioisotope-based brachytherapy or conventional clinical accelerator-based IMRT (Intensity Modulated Radiation Therapy) can produce conformed irradiation volumes, but they are not used for micro-conforming volumes because first, such small clusters of tumors cannot be localized (see point #1), second, because even if point #1 were met, several catheters would each have to be positioned or several independent treatments would be required to irradiate irregular, non-contiguous volumes. Accordingly, today's methods to treat tumor margins irradiate a homogeneous layer of tissue that contains more normal tissue than tumor resulting in either recurrence if the prescribed dose is too low to preserve normal tissue function or normal tissue sequelae if the prescribed dose is too high to prevent tumor regrowth. Moreover, today's irradiation treatments require specialized suites in order to shield personnel from the high-energy irradiation.

With respect to point #3, the closest method that exists today to find and treat residual tumor cells in tumor margins is known as radio-guided surgery. Following surgical resection of the tumor and radiotracer administration into the patient, a detector device is placed into the cavity, ‘hot’ areas corresponding to remnant tumors in the tumor bed are detected, noted to memory (the surgeon's), shaved out, and repeated until no more is detected or no more normal tissue can be jeopardized. The inherent inaccuracies in the current State-of-the-Art method compromise optimal treatment.

In conventional radio-guided surgery, first, the detector is used to find “hot” areas, which indicate tumor, second, the detector is removed from the surgical wound, and third, the surgeon resects the “hot” area from memory of where the “hot” spot was detected. Our invention would allow identification of the “hot” tumor area and irradiation of the tumor without having to move the detector from the site so that there is no imprecision of location. Additionally, our device will be able to image smaller and deeper tumors with higher precision than any currently available imaging technique. And because of its portability and limited shielding requirements, our device can be moved to any room. Further, because of the well-characterized physics of the therapeutic x-rays emitted by our device, the precision in getting the entire tumor will be superior to manual resection—without compromising integrity of the surrounding normal tissue. The low-energy x-rays produced by our device can be easily shaped and intensity-modulated using windows filled with high-z material (e.g., iodinated radiographic contrast agent) fluids that could be pumped in and out according to the depth of tissue penetration required as assessed from the acquired tomographic images also by our device.

Also in the existing practice of interstitial or intracavitary radiation therapy, where a miniature x-ray device is currently used, the tumor is imaged elsewhere, marked (e.g., tattooed) with reference to anatomical and artificial landmarks, and irradiated according to such “historical” guides. Our invention would allow real-time guiding to the living tumor because only viable cells will uptake the positron-emitting isotopic compound and because the intra-corporeal component (ICC) probe will remain in the same position relative to the tissue cavity; no misalignment for targeted radiation will occur. Moreover, the current practice of irradiating the tumor bed after surgical resection to destroy residual neoplasia, uses a balloon inflated around the miniature x-ray device within the surgical cavity to irradiate a homogeneous annular margin in an otherwise irregularly-shaped heterogeneous tissue, which limits the depth that can be irradiated without sacrificing normal tissues. Our invention would obviate this problem because only margin areas that show tumor can be irradiated more aggressively facilitated by the high-z material pumped windows as described earlier.

Specifically with respect to imaging early stage intracavitary or intraluminary tumors, our invention is superior to any currently used state-of-the-art device. The imaging mode of HRAPPET-IMRT (aka HRAPPET) is a novel variation of positron emission tomography (PET). As a portable device, HRAPPET would be used like an ultrasound unit, but possesses superior specificity and sensitivity by employing PET principles to detect radiotracers taken up by tumors. It is distinct from conventional beta/gamma-detecting probes in that it produces tomographic images for precise 3D localization like conventional PET, but differs from conventional PET by utilizing a novel, non-conventional geometry to improve resolution by at least an order of magnitude compared to conventional PET.

In summary, the specific novel features are: 1) a tomographic imaging system with an asymmetric configuration of the annihilation detectors, one inside and one outside the patient body, that produce unrivaled resolution and specificity 2) intensity modulation of the therapeutic x-rays using iodinated contrast agent (or any high-Z material liquid), and 3) the marriage of #1 and #2 to produce a device capable of simultaneous imaging and treatment of sub-millimeter-sized tumor clusters.

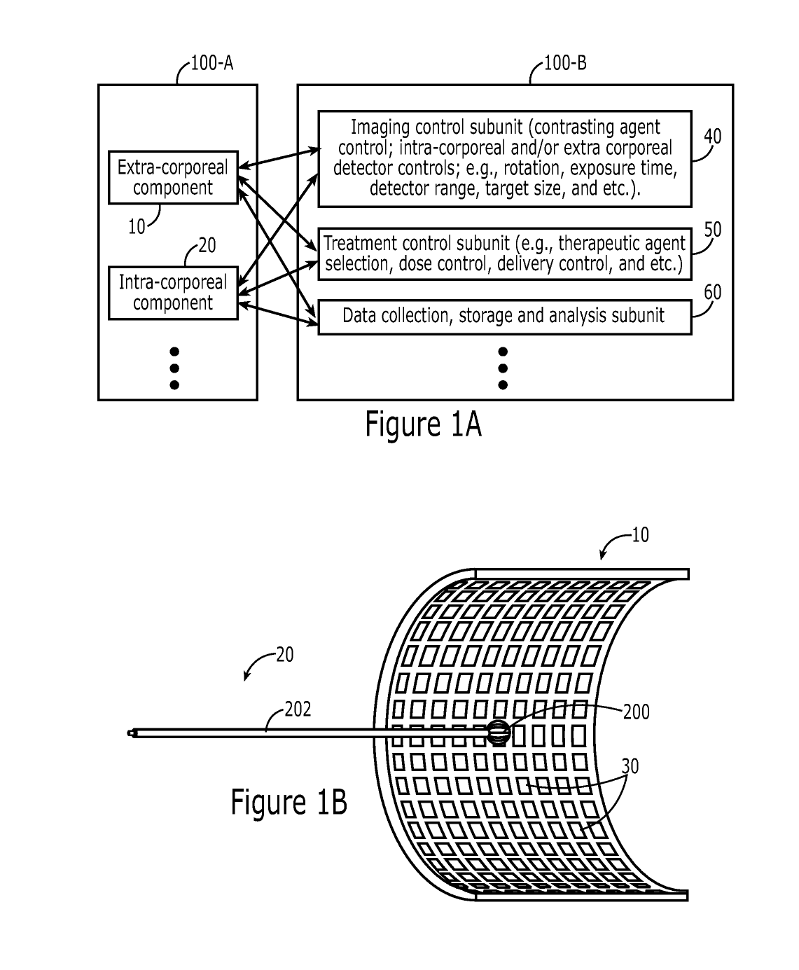

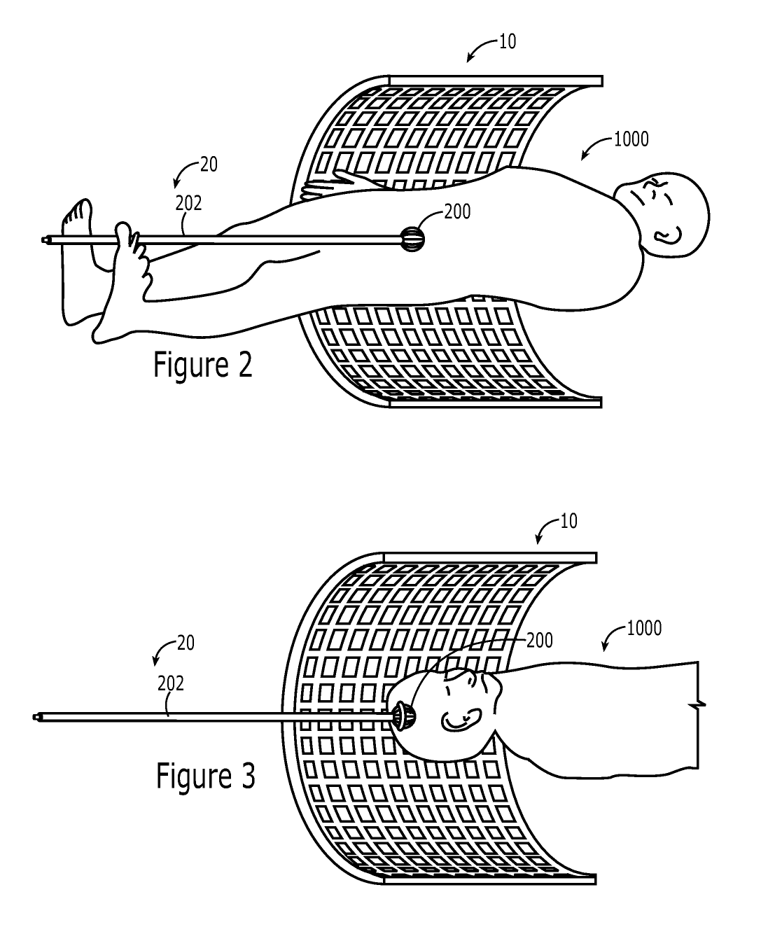

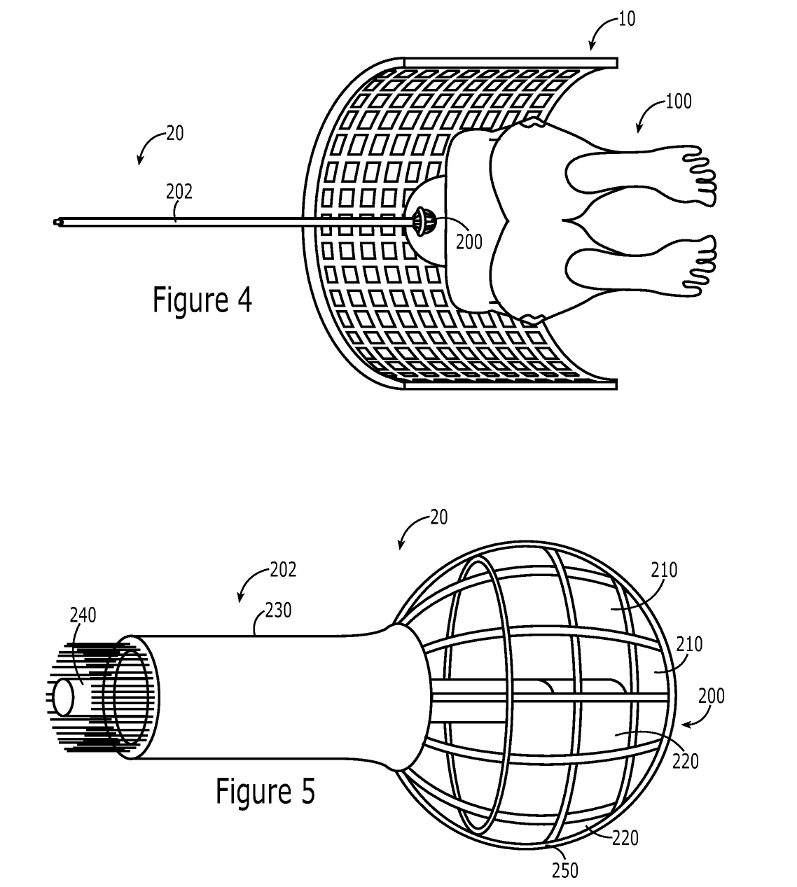

The basic configuration of our invention is shown in FIGS. 1A and 1B. The key novel parts of the device are the extra-corporeal component (ECC) and ICC. The ECC is an arc slab array of gamma detectors, which is positioned outside the patient. The ICC is a small probe that is placed inside the patient's body within anatomical or surgical cavities as shown in FIGS. 2, 3, and 4.

FIG. 1A depicts an exemplary realtime imaging and therapeutic system comprising two main parts: 1) an imaging/therapy unit 100-A including an extra-corporeal component 10 and an intra-corporeal component 20 and 2) a monitoring/controlling/processing unit 100-B including an imaging control subunit 40, a treatment/therapy control subunit 50, and a data collection, storage and analysis subunit 60. The categorization here is mainly for illustration purposes and should not be contused in any way to limit the scope of the invention.

Referring to FIG. 1B, an exemplary imaging/therapy system is depicted including an intra-corporeal component 20 and an extra-corporeal component 10. Extra-corporeal component 10 comprises one or more detectors 30. In some embodiments, the extra-corporeal component 10 comprises one or more types of detectors. In some embodiments, each of the one or more detectors 30 comprises a plurality of detectors each capable of detecting and recording one or more signals from a subject such as a human patient. In some embodiments, the detectors detect the same type of signal. In some embodiments, the detectors detect different types of signals.

In some embodiments, the detectors included in the extra-corporeal component are gamma detectors. For example, the detectors are used for position-annihilation detection based on coincident gamma rays. In some embodiments, a probe is administered (e.g., a fluorescent probe or radioactive probe), and the detectors are suitable for detecting fluorescent and/or radiation signals.

In some embodiments, the plurality of detectors comprises 10 or more detectors, 20 or more detectors, 30 or more detectors, 40 or more detectors, 50 or more detectors, 60 or more detectors, 80 or more detectors, 100 or more detectors, 120 or more detectors, 140 or more detectors, 160 or more detectors, 180 or more detectors, 200 or more detectors, 250 or more detectors, 300 or more detectors, 350 or more detectors, 400 or more detectors, 450 or more detectors, 500 or more detectors, 600 or more detectors, 700 or more detectors, 800 or more detectors, 900 or more detectors, 1,000 or more detectors, 1,200 or more detectors, 1,400 or more detectors, 1,600 or more detectors, 1,800 or more detectors, 2,000 or more detectors, 3,000 or more detectors, 5,000 or more detectors, 10,000 or more detectors, 20,000 or more detectors, 30,000 or more detectors, 50,000 or more detectors, or 100,000 or more detectors.

In some embodiments, the one or more detectors detect signals from a particular region of the patient, for example, the head, the neck, the chest, the stomach, the colon, the cervix, the ovaries, a blood vessel and etc.

In some embodiments, the detectors are jointly arranged in a detector array. In some embodiments, the detector array is curved around a target area in a patient, for example, as depicted in FIGS. 1-4. In some embodiments, the detector array forms an enclosure surrounding the target area; for example, a ring-like detector array. In some embodiments, the detector array has the capacity to detect signals from a specific region of the patient, for example, the head, the neck, the chest, the stomach, the colon, the cervix, the ovaries, and etc. In some embodiments, the detector array has the capacity to detect signal from the entire body of a patient.

The extra-corporeal detector array can be arranged in any orientation relative to the target detection area in a patient's body; see, for example, FIGS. 2-4.

In some embodiments, the intra-corporeal component 20 comprises an intra-corporeal unit 200 and a connecting unit 202. Intra-corporeal unit 200 is actually being placed inside a patient's body. Connecting unit 202 connects the intra-corporeal unit 200 with other parts of the system, including, for example, the external monitoring/controlling/processing unit 100-B such as computers and storage media.

It is to be noted that in the exemplary embodiments, as depicted in FIGS. 1-4, intra-corporeal unit 200 is connected to a straight connecting unit 202. However, connecting unit 202 can be curved or made of flexible material without any limitations to its functionalities.

The intra-corporeal unit 200 is actually being placed inside a patient's body. In some embodiments, intra-corporeal unit 200 is placed in a cavity within patient 1000. For example, as depicted in FIG. 2, intra-corporeal unit 200 is placed within the cervical cavity of a patient. In FIG. 3, intra-corporeal unit 200 is placed in a cavity of the skull of a patient.

Intra-corporeal unit 200 can be made of any metal, glass, or hypo-allergic synthetic materials. In some embodiments, a titanium-based device can be used.

The size and shape of the intra-corporeal unit 200 can vary with respect to its intended purposes. For example, an intra-corporeal unit 200 on the order of centimeters or smaller can be used for brain tumor detection and treatment while a subunit of a few centimeters or larger can be used for treatment of the digestive system, such as colon or stomach and the treatment of the breast or uterus. One of skill in the art can select the size of an intra-corporeal unit 200 based on the location of a possible disease target. Without limitation, the longest dimension of an intra-corporeal unit 200 can be 1 centimeter or smaller, 2 centimeters or smaller, 3 centimeters or smaller, 4 centimeters or smaller, 5 centimeters or smaller, 7 centimeters or smaller, 8 centimeters or smaller, 9 centimeters or smaller, 10 centimeters or smaller, 12 centimeters or smaller, 15 centimeters or smaller, 20 centimeters or smaller, or 25 centimeters or smaller.

In some embodiments, an intra-corporeal unit 200 is symmetrical, spherical or near-spherical in shape and has a smooth surface. In such embodiments, the cross-sectional surface along the symmetrical axis is circular or near circular. In some embodiments, an intra-corporeal unit 200 has a shape similar to that is an America football or an egg; for example, it has an oval or oval like surface when a cross-section is taken along its longest axis. In some embodiments, an intra-corporeal unit 200 has a symmetrical shape with smooth surface. In some embodiments, an intra-corporeal subunit has a non-symmetrical shape. In some embodiments, an intra-corporeal unit 200 has a non-smooth surface.

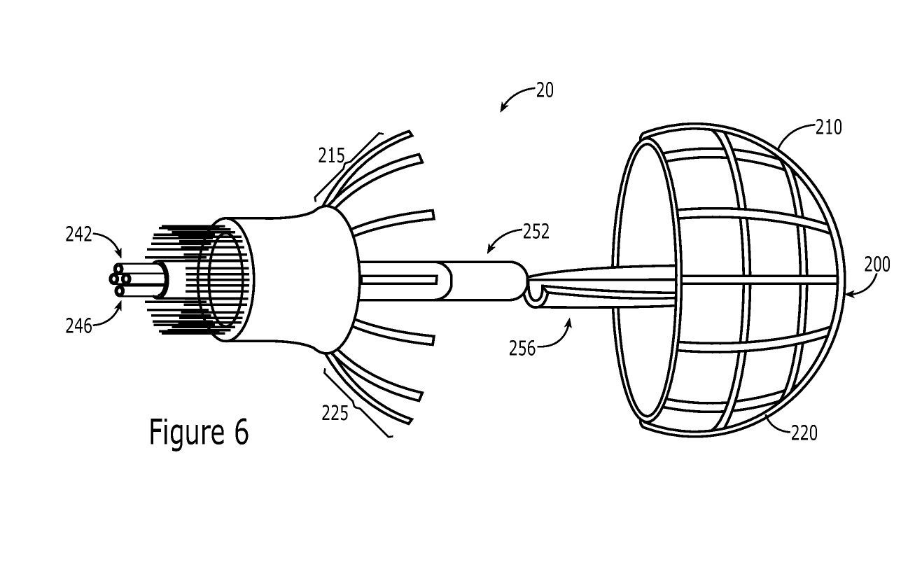

The ICC is the marriage of an array of gamma detectors, a miniature x-ray source, and a set of hollow miniature windowpanes into which iodinated (or other high-z material) radiographic contrast medium (IRCM) of various iodine concentrations can be pumped. (see FIGS. 5 and 6) The gamma detectors of the ICC are electronically coupled to those of the ECC for detection of coincident annihilation photons from positron-emitting tracers administered to the patient. This asymmetric configuration is novel and allows for detection and 3D image reconstruction of sub-millimeter-sized tumors via PET principles leading to high-resolution tomographic images.

FIG. 5 depicts a close-up view an exemplary intra-corporeal unit 200 being connected with an exemplary portion of a connecting unit 202. In some embodiments, intra-corporeal unit 200 comprises a treatment subunit 210 (e.g., a x-ray source, a radioactive agent, a chemotherapeutic agent or other anti-cancer reagents) and a detector/imaging subunit 220 (e.g., one or more gamma detectors). In some embodiments, the treatment subunit 210 and detector/imaging subunit 220 are enclosed in an outer shell 250. In some embodiments, the treatment subunit 210 and detector/imaging subunit 220 can be rotated with respect to the patient. In some embodiments, the treatment subunit 210 and detector/imaging subunit 220 can be rotated with respect to the extra-corporeal components. In some embodiments, the outer shell 250 does not rotate with the treatment subunit 210 and/or detector/imaging subunit 220.

In some embodiments, treatment subunit 210 comprises iodine solution compartments for modulating the intensity of x-rays from the x-ray source. In some embodiments, iodine solutions in different compartments have different concentrations. Iodine solutions in the form of a radiographic contrast agent are used to modulate the intensity of the emanating x-rays and thus are not intended to be released into the tissue. In some embodiments, the iodine can be target-injected, in which case the x-ray dose can be enhanced at the injection site. In such an embodiment, a distinct mechanism for a retractable needle will be required to be incorporated into the unit and we have not designed such a device. In some embodiments, iodine solution can be released from a compartment in a controlled manner over a period of time. FIG. 5 depicts the treatment subunit 210 and detector/imaging subunit 220 as top hemisphere and bottom hemisphere, respective. However, one of skill in the art will understand that the treatment subunit 210 and detector/imaging subunit 220 can be configured in any manner; for example, each subunit can have smaller units that are inter-dispersed in alternating fashion. Preferably, both treatment subunit 210 and detector/imaging subunit 220 have equal or similar access to a target area. Additional information regarding x-ray dose modulation using iodine can be found, for example, in Iwamoto K S et al., 1987, “Radiation dose enhancement therapy with iodine in rabbit VX-2 brain tumors,” Radiother Oncol. 8(2):161-170; Iwamoto K S, et al., 1990, “The CT scanner as a therapy machine,” Radiother Oncol. 19(4):337-343; Norman A, et al., 1991, “Iodinated contrast agents for brain tumor localization and radiation dose enhancement,” Invest Radiol. 26 Suppl 1:S120-1; discussion S125-12; Solberg T D et al., 1992, “Calculation of radiation dose enhancement factors for dose enhancement therapy of brain tumours,” Phys Med Biol. 37(2):439-443; and Iwamoto K S, et al., 1993, “Diagnosis and treatment of spontaneous canine brain tumors with a CT scanner,” Radiother Oncol. 26(1):76-78; each of which is hereby incorporated by reference in its entirety.

In some embodiments, treatment subunit 210 delivers and releases therapeutics to a target identified by the extra-corporeal and intra-corporeal detectors. In some embodiments, treatment subunit 210 can place an drug implant adjacent to or at a target identified by the extra-corporeal and intra-corporeal detectors to achieve sustained delivery of one or more therapeutic agents. In some embodiments, treatment subunit 210 includes a microsurgical device that can surgically remove a target identified by the extra-corporeal and intra-corporeal detectors. In some embodiments, a combination of treatment methods can be used. For example, a microsurgical device can be used to remove a diseased tissue (e.g., a tumor) before radiation or other therapeutics are applied. Also for example, an incision can be made in a diseased tissue before an implantable drug delivery device is implanted for sustained drug delivery and treatment. Existing microsurgical tools can be implemented where suitable. For example, microsurgeries are more applicable with a larger body cavity. In some embodiments, optical fiber is used to provide illumination. In some embodiments, it is possible to sequentially apply different treatment methods. For example, microsurgical tools are used before they are replaced with an x-ray source for radiation treatment. Exemplary microsurgical devices that can be used include but are not limited to those available through Microsurgical Technology, Inc. (Redmond, Wash.). In some embodiments, modifications can be made to suit a particular purpose.

In some embodiments, connecting unit 202 comprises an outer casing 230 and an inner casing 240 (e.g., as illustrated in FIG. 5). In some embodiments, the outer casing 230 is connected with intra-corporeal unit 200 (e.g., treatment subunit 210) via a plurality of inlet and/or outlet tubes 215 for solutions of therapeutic or modifying agents such as iodine, one or more other contrast agents, one or more chemotherapeutic agents, one or more anti-cancer drugs, and etc. In some embodiments, the outer casing 230 is connected with intra-corporeal unit 200 (e.g., detector/imaging subunit 220) via a plurality of detector leads 225.

In some embodiments, intra-corporeal unit 200 includes a cavity into which one or more therapeutic agents or treatment sources/devices can be placed. For example, at depicted in FIG. 6, a radiation source such as an X-ray source 252, shielded by an radiation shield 256, is placed in the cavity in intra-corporeal unit 200. In some embodiments, one or more inlets and/or outlets 242 delivering coolants for the x-ray source are placed in inner casing 240. In some embodiments, one or more anodes and/or cathodes for high voltage leads of the detectors are placed in inner casing 240. In some embodiments, one or more inlets and/or outlets 242 delivering coolants for the x-ray source and one or more anodes and/or cathodes for high voltage leads of the detectors are both placed in inner casing 240.

In some embodiments, electrical and/or mechanical controls for the treatment subunit 210 and/or detector/imaging subunit 220 are placed in inner casing 240. In some embodiments, electrical and/or mechanical controls for the treatment subunit 210 and/or detector/imaging subunit 220 are placed between inner casing 240 and outer casing 230. In some embodiments, electrical and/or mechanical controls for the treatment subunit 210 and/or detector/imaging subunit 220 are embedded in inner casing 240. In some embodiments, electrical and/or mechanical controls for the treatment subunit 210 and/or detector/imaging subunit 220 are embedded in outer casing 230. In some embodiments, electrical and/or mechanical controls for the treatment subunit 210 and/or detector/imaging subunit 220 are embedded in inner casing 240 and outer casing 230.

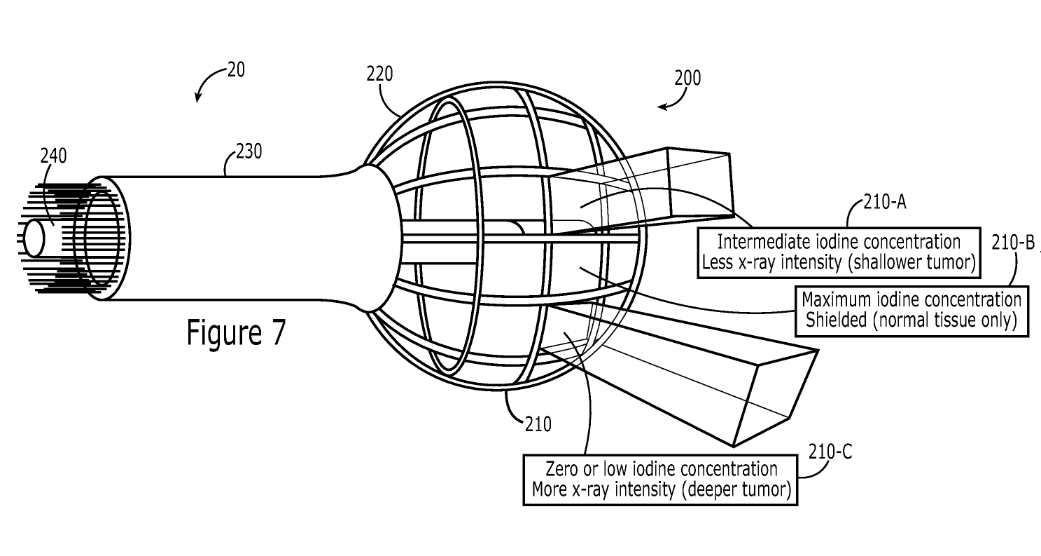

In some embodiments, the radiation source 252 further comprises an inlet and an outlet for a contrast agent. In some embodiments, the contrast agent (e.g., an iodinated agent, a high-Z material liquid, and a combination thereof) of various concentrations can be pumped into opening on the compartment of treatment subunit 210 to modulate the intensity of radiation (e.g., FIG. 7). The density modulation correlates with the location of a diseased target (e.g., a tumor). For example, intermediate iodine concentrations are combined with less radiation intensity (e.g., x-ray) for treatment of shallower tumor. Zero or very low iodine concentrations are combined with more radiation intensity (e.g., x-ray) for treatment of deeper tumor. Maximum iodine concentrations are used to fully reduce radiation intensity (e.g., x-ray) for protection of normal tissues.

The miniature x-ray tube produces 50 kVp (or more) x-rays for radiotherapy of the detected tumors at least 2 cm deep. The iodine-windows act like the multi-leaf collimators of a conventional IMRT by modulating the intensity of the x-rays exiting the ICC. The iodine concentration of the IRCM can be used undiluted or diluted to adjust for depth dose into the tissues—leading to localized conformal therapy based on the high-resolution imaging from its partner detection system (see, e.g., FIG. 7) Although iodine was used in the description of the method of x-ray intensity modulation because it is effective, we have experience with it, and it is prevalent in clinical use, other high-Z materials can also be used.

In some embodiments, the system/apparatus descried herein can be used as a stand-alone high resolution imaging device for detection of early-stage tumors that currently have no other method of reliable detection such as for ovarian cancer. This lack of reliable detection of ovarian cancer is the major reason for deaths attributed to this disease—by the time the tumor is diagnosed, it is very late. The system and apparatuses described herein have the necessary sensitivity and specificity to image sub-millimeter tumors by insertion of the ICC probe into the uterus. This ability would be a great leap forward in the screening of such diseases.

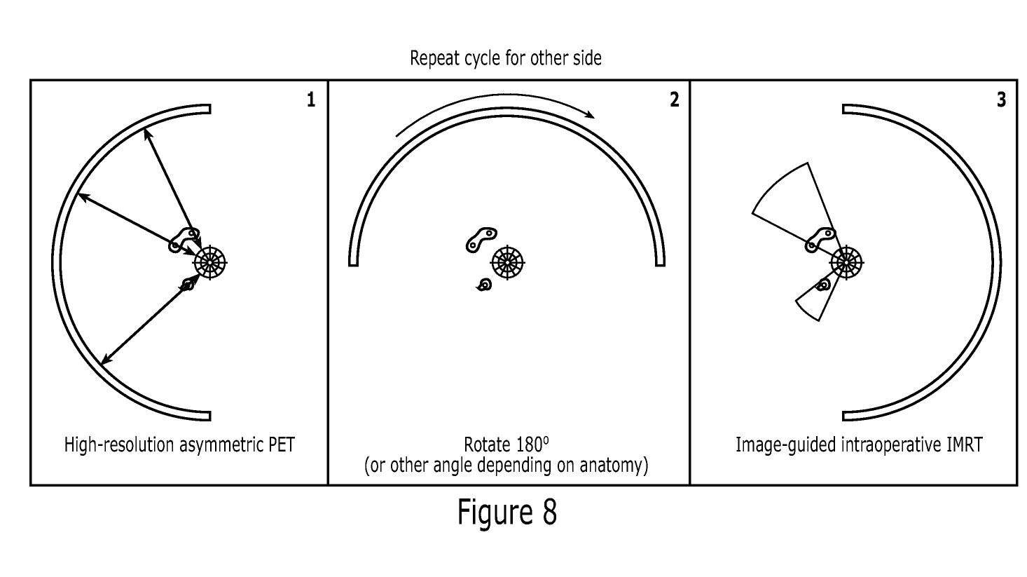

Additionally, the system/apparatus descried herein can be used as a near-realtime imaging and conformal radiotherapy device. FIG. 8 illustrates the sequence of steps for the operation of an exemplary system such as the HRAPPET-IMRT. In the first step, the system is used as a screening device. After the patient is administered a positron-emitting-isotope-labeled compound (like 18-F-deoxyglucose or any future molecular marker that would identify specific tumors or diseased tissue) that is preferentially taken up by tumor cells, the ICC is inserted into the surgical cavity. Half the cavity will be imaged, the ICC will be rotated along with the ECC, which always faces the imaging hemisphere of the ICC, the reconstructed image will be assessed for residual tumor left behind in the margin by the surgeon, and only the volumes containing tumor will be irradiated.

The systems and methods described herein offer numerous advantages. In one aspect, using intra-corporeal and extra-corporeal detectors simultaneously or within a short period to each other allows a diseased target tissue or organ (e.g., a tumor or an ulcer) to be more precisely located. In another aspect, accuracy can be further improved when multiple detections can be made from different orientation; e.g., both intra-corporeal and extra-corporeal detectors can be rotated for this purpose. Moreover, treatment can be delivered at the same time when a diseased target tissue or organ (e.g., a tumor or an ulcer) is identified or shortly thereafter, which enhances efficiency and accuracy. Further, it is possible to offer multiple types of treatment depending on the nature of the diseased tissues, from surgical removal, radiation treatment to sustained drug treatment, or a combination thereof.

Unique algorithms for image reconstruction and dose calculations will be combined with treatment planning software to facilitate the operation of simple mini-pumps (not shown in figures) that will fill each windowpane with the required concentration of IRCM to modulate the dose and dose volume according to the radiation oncologist's prescribed tumor volume. This will obviate unnecessary irradiation of normal tissue not containing any tumor and will therefore allow for increase dosing and better tumor control. Current protocols are incapable of such ‘dose painting’.

Finally, because the x-rays produced by the device are low energy, heavy shielding is unnecessary during radiotherapy, making it a portable device that can be used in any conventional surgical environment.

Systems, devices, and methods described herein are suitable for imaging and treatment of sub-millimeter-sized tumors that are embedded in tissues; e.g., at depths greater than 1-2 mm, greater than 2-3 mm, greater than 3 mm, greater than 4 mm, greater than 5 mm, greater than 6 mm, greater than 7 mm, greater than 8 mm, greater than 9 mm, greater than 10 mm, greater than 12 mm, greater than 15 mm, or greater than 20 mm.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

Examples presented here focus on radiation treatment of tumor by an x-ray source with designs and reagents suitable for this purposes. However, it should be emphasized that alternative designs and reagents can be made for different diseased targets. They can also vary with the locales of the diseased targets.

Preliminary in vitro experiments using human cell lines were performed showing the radiobiological effectiveness of the miniature x-ray source compared to conventional clinical megavoltage irradiation machines. Monte Carlo simulations were done on the variability of percent tissue depth dose depending on filtration of the x-rays. The maximum-likelihood expectation maximization algorithm image reconstruction analysis was used to demonstrate the high resolution potential for this invention.

### Example 1

### Miniature X-Ray Tube Characteristics

Figure A displays the photon spectrum in 5 cm of air, 1.5 cm of water, and 5 cm of water from the source operated at 50 kVp; note the substantial hardening of the beam with distance and bolus. The depth dose characteristics are illustrated in figure B for the source operated at 20 kVP and at 50 kVp with or without bolus. These data suggest that via collimation, filtration, and/or energy modulation, treatment conformation to the desired volume is possible.

### Example 2

### Relative Biological Effectiveness (RBE) of the Source

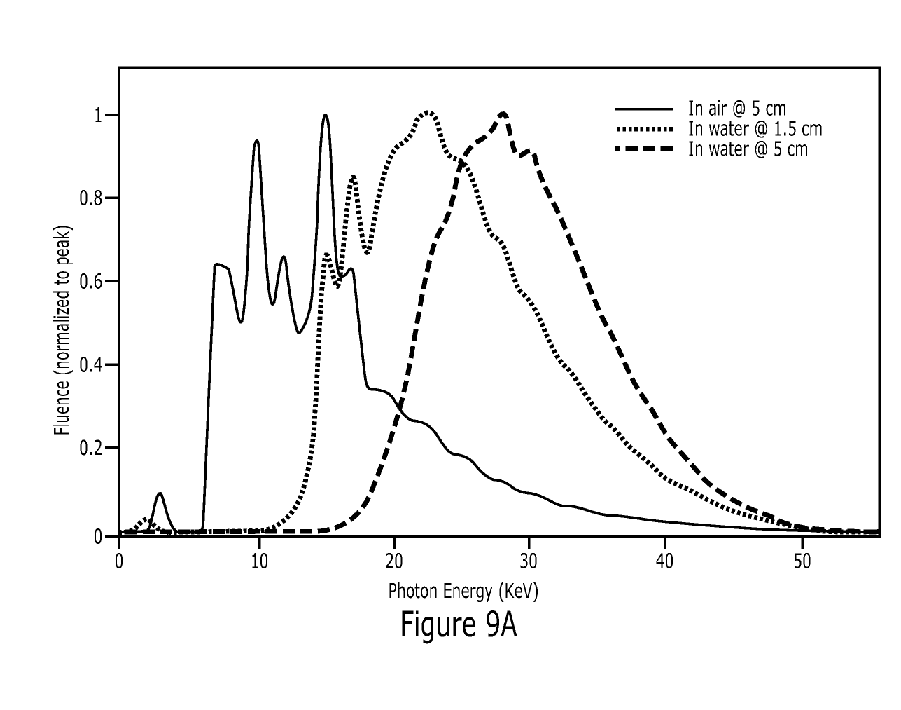

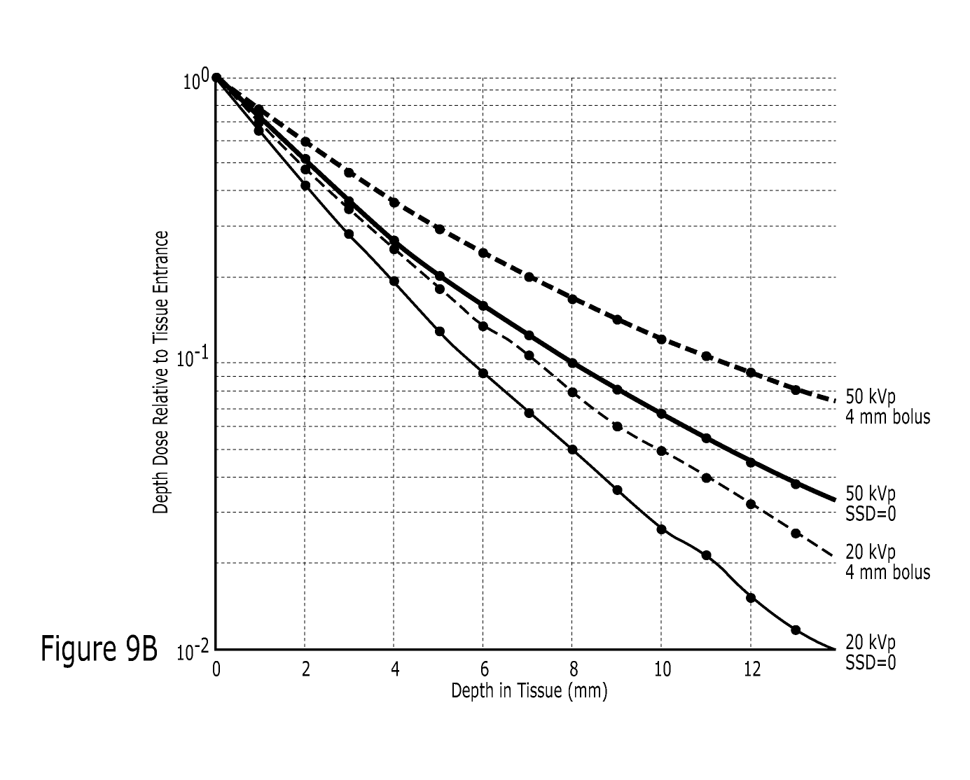

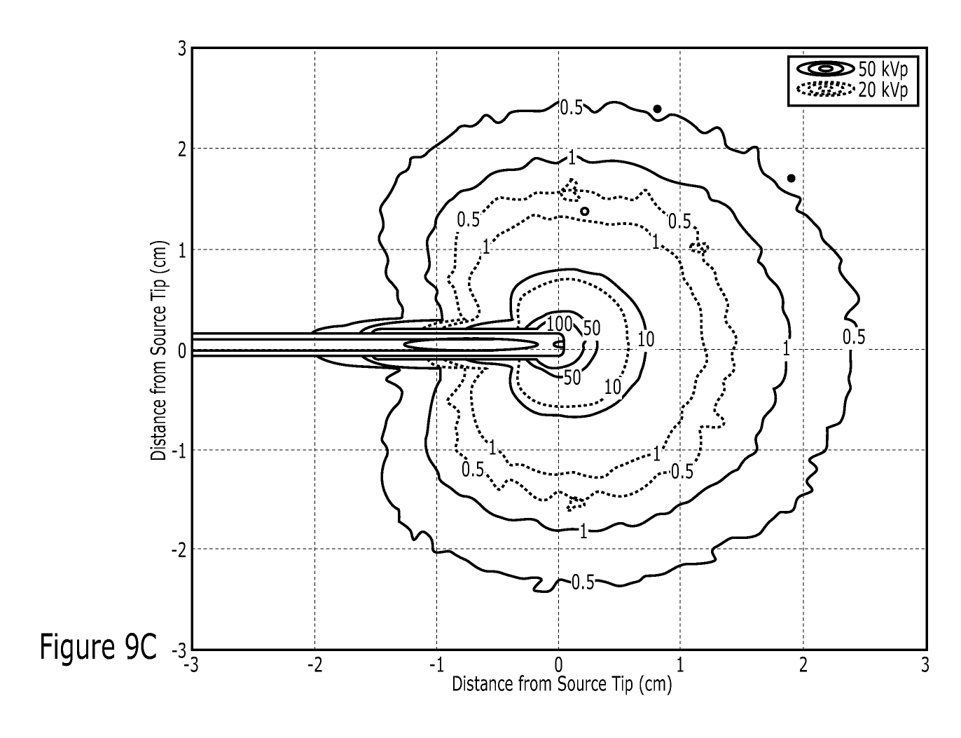

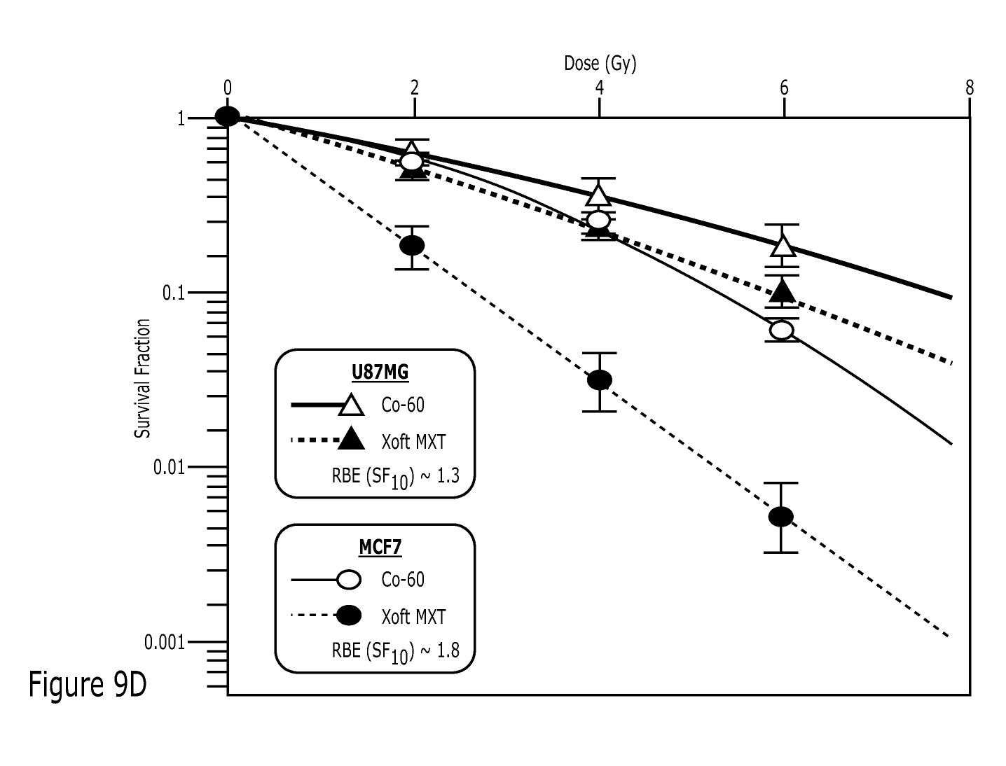

We have measured preliminary RBEs of the x-rays from the source using U87MG human glioblastoma multiforme (GBM) and MCF7 human breast cancer cells in a standard clonogenic survival assay method. Compared to cobalt-60 irradiation for 10% survival, the RBEs are 1.3 and 1.8, respectively. (FIG. 9D) Even with a highly radioresistant GBM cell line, we show an RBE value greater than 1. The high surface dose deposition and >1 RBE of orthovoltage x-rays has always been a hindrance for EBRT of non-superficial tumors, but they become a clear advantage for the Axxent system in managing disease interstitially, intracavitarily, and intraoperatively. Sparing of normal tissues can be achieved by the rapid depth-dose fall-off (FIGS. 9B & 9C). Use of iodine for dose enhancement in the tumor and dose reduction in the normal tissue should further improve the therapeutic ratio; this study is still currently in progress.

Characterization data of source are shown in FIGS. 9A-9D: 9A) X-ray spectra in air or water; 9B) Relative depth dose in water for two beam energies and with or without bolus as calculated by Monte Carlo simulation; 9C) Two-dimensional representation of ‘B’; and 9D) Clonogenic survival of U87MG human GBM cells and MCF7 human breast cancer cells for derivation of RBE compared to a cobalt-60 irradiator.

### Example 3

### A Pilot Ambi-Cranial PET System for GBM Surgery Guidance: Characterization and Analysis

### I. Introduction

Gliobastoma multiforme (GBM) is the most common and most aggressive malignant primary brain tumor in humans. GBMs are distinguished by extensive and diffuse infiltration of tumor cells into the dense network of interwoven neuronal and glial processes rendering these tumors extremely difficult to excise without large concomitant areas of normal brain.

To best identify and excise tumor during the surgical operation, many image-guided systems have been introduced, such as a stereotactic navigation system (SNS) that combines a microscope with a tracking system and the use of pre-operative MRI images to relay the 3D location of the scalpel relative to the tumor and brain structures in real-time. However, both systems suffer from brain-shift during and following removal of the gross tumor mass. Intraoperative MRI systems are costly and require MRI-compatible surgical instruments.

This project aims to develop a system to detect tumor and localize activity in the surgical frame of reference, known as radioguided surgery (RGS). Section II introduces the general notion of the ambi-cranial PET system under development and reports the simulation and image reconstruction procedures. Section III presents the analyzed system behavior and Section IV discusses their implications for clinical utility.

### II. System Configuration, Simulation and Image Reconstruction

A. System Configuration

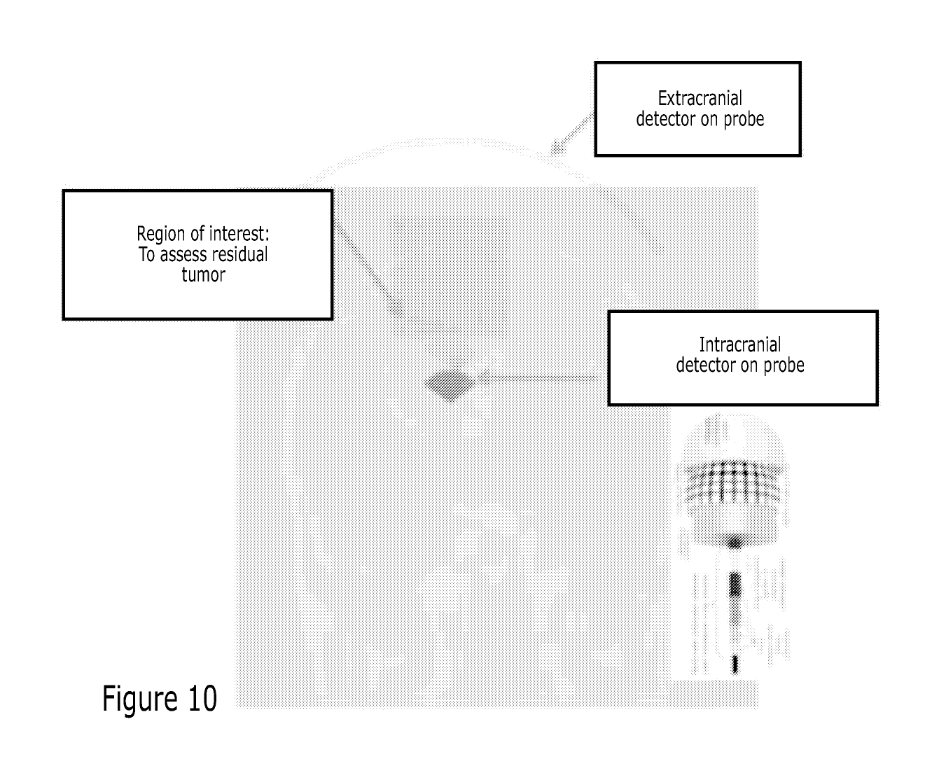

Upon contrast injection, a probe with dual treatment and photon detection functionality will be inserted into the surgical cavity. In the exterior of the skull, an arc detector array covers the “opposite” side of the ROI in the extracranial space. The goal is to detect and estimate pair production activities in the ROI which resides between the probe and the skull, by identifying coincidence events detected by the probe and the arc detectors, as illustrated in FIG. 10.

B. Simulation Process
Simulation was configured in 2D plane. The probe and arc detectors were simulated by 20° —2 mm detector units and 44°—6 mm detectors units respectively, and placed 220 mm apart from each other along the radial direction. A region of interest of size 220 mm°—40 mm was simulated where activity were to be estimated.

The system matrix was generated with conventional geometrical Siddon ray-tracer (S-RT) method [1]. An attenuation map was simulated to account for realistic absorption and scattering. Variation in detector efficiency was simulated to yield the nominal detected coincidence. The final measurement was simulated according to Poisson statistics based on the nominal detection counts.

The overall simulation follows [2], based on the following:
Yᵢ˜Poisson{Σ^{p}ⱼ₋₁aᵢⱼλⱼ^{true}+rᵢ+sᵢ}, where aᵢⱼ>0 is entry of the system matrix A incorporating scan geometry, attenuation, detector efficiency, etc. λⱼ^{true}≧0 is the activity at voxel j; and rᵢ≧0 and sᵢ≧0 are the means of accidental coincidence events and scatters.

C. Reconstruction

The maximum-likelihood expectation maximization algorithm [3] was used for image reconstruction.

### III. Results

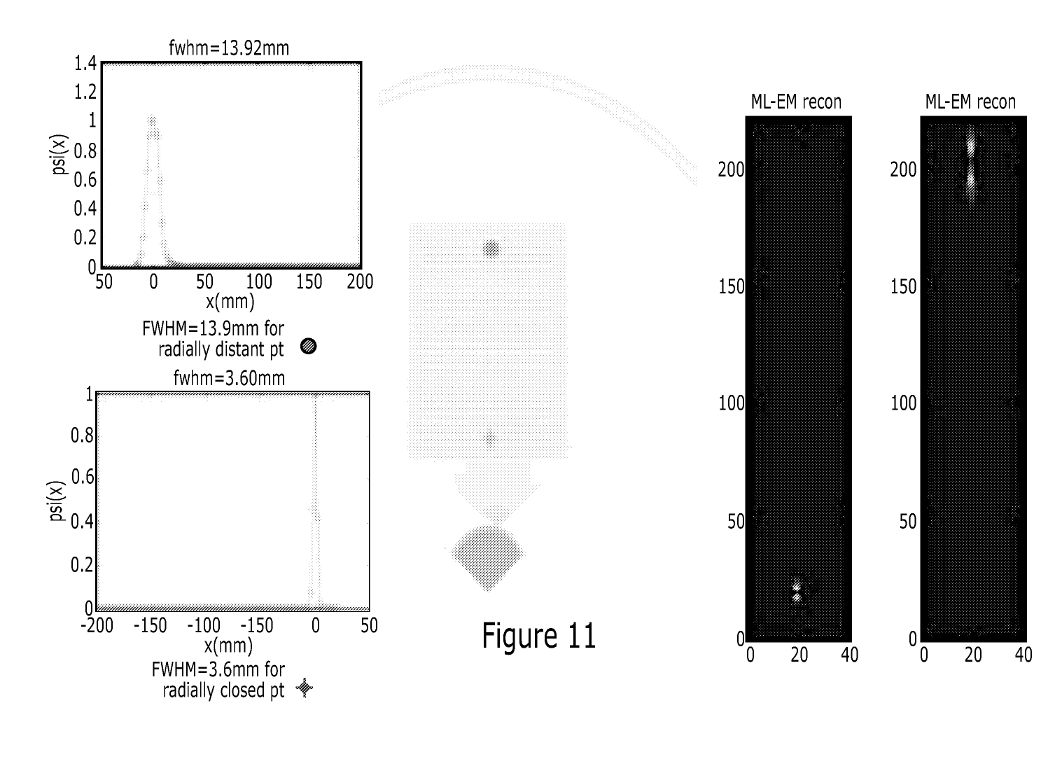

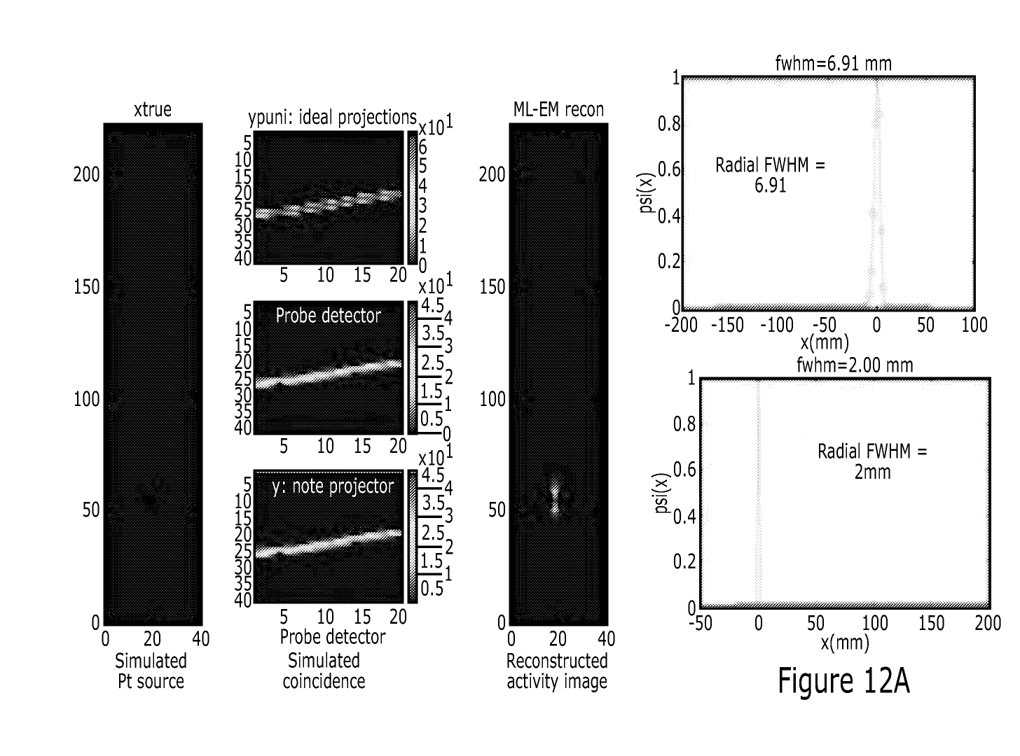

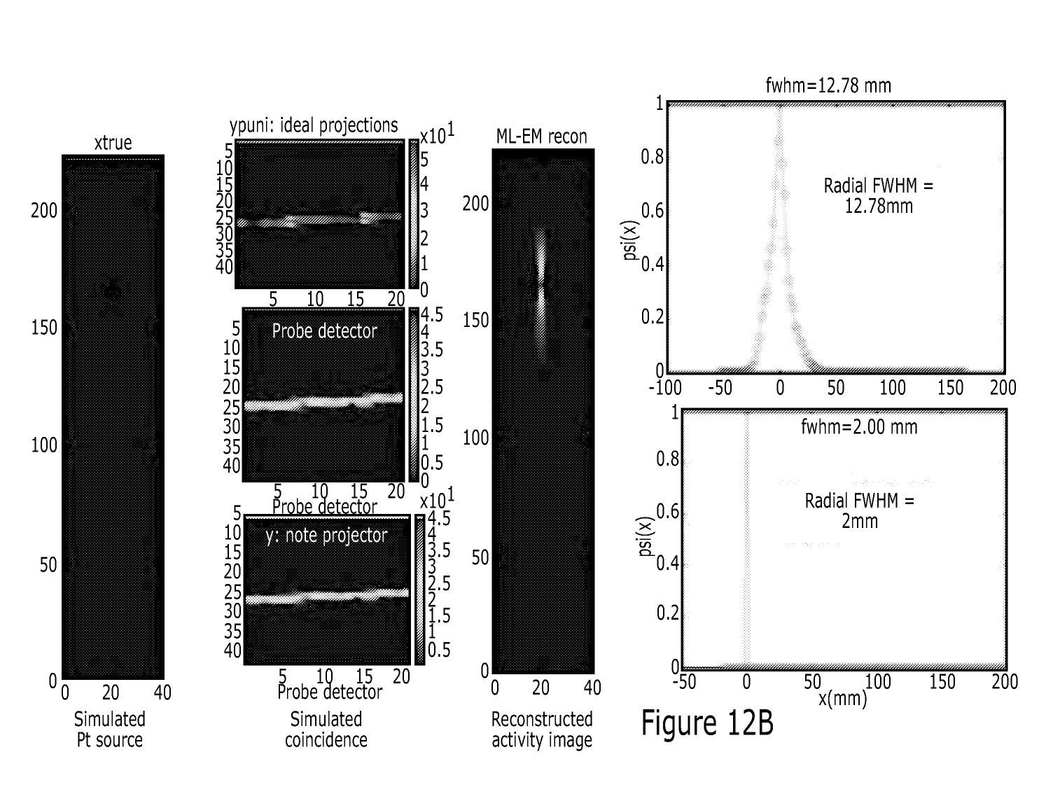

The special asymmetric geometry of this system determines the unique system matrix. Spatial varying responses are depicted in FIG. 11 and FIGS. 12A and 12B.

Table I quantitatively illustrates the general trend of decreasing spatial resolution as the estimation point moves away from the surgical cavity towards the exterior of the skull.

TABLE I
Reconstruction resolution as a function of radial
distance from the intracranial probe.

| Radial Distance (mm) | Radial FWHM | Tangential FWHM |
|---|---|---|
| 6 | 2.17 | 2 |
| 20 | 3.6 | 2 |
| 74 | 6.91 | 2 |
| 100 | 10.56 | 2 |
| 120 | 9.84 | 2 |
| 140 | 13.67 | 2 |
| 164 | 12.78 | 2 |

These results suggest among others the following observations: 1) reconstruction is inhomogeneous and anisotropic; 2) the directional resolution along the radial direction varies significantly. In particular, reconstruction closer to the probe detector enjoys superior resolution close to the voxel size, while activities closer to the exterior skull can only be reconstructed with low resolution along the radial direction; and 3) the directional resolution in the tangential direction is almost constant throughout the whole region of interest. By radial symmetry, it is reasonable to conjecture that the whole domain has uniformly high tangential resolution.

### IV. Discussion and Conclusion

The goal of this work was to characterize the detection capability of the ambi-cranial PET system and assess its potential utility to detecting and localizing residual lesion in surgical excision procedure to remove GBM tumor, or to guide the radiotherapy beam on the probe towards the proper direction. Intriguingly, the specific pattern of spatial inhomogeneity and anisotropicity in reconstruction resolution aligns with such goals. In particular
When the system is used to aid surgical resection of GBM lesion, the reconstruction result is used to inform the surgeon whether there are residual lesion tissue at the immediate vicinity of the surgical cavity where the mass tumor has been removed. In this case, accurately reconstructing near-field activity is of the utmost importance, which is satisfied by the good near-field reconstruction resolution as in FIG. 3(a). Moreover, as the surgeon incrementally removes more tissue and enlarges the cavity, the probe effectively pushes the cavity frontier towards the skull and changes the reconstruction coordinate. As a consequence, activity at the interior surface of the ROI adjacent to the surgical cavity can always be reconstructed with high resolution—providing information as to whether these regions should be further removed.
When the intracranial probe operates as a radiotherapy unit, deciding irradiation direction is most critical. Fortunately, tangential resolution is uniformly high throughout the ROI, providing guidance for beam steering with high confidence.

In summary, this pilot simulation and image reconstruction study has revealed the unique detection and estimation characteristics of the ambi-cranial PET system under development. Preliminary results suggest the potential utility of this system to satisfy clinical needs for surgical guidance (both conventional and radiological), despite the low radial resolution in the far field. Upon further validation with higher resolution and alternative configuration parameters, results reported here will be used to design and construct the physical detectors. To provide fast feedback in clinical environment and minimize imaging dose, reconstruction speedup utilizing ordered subset [4] type technique and parallelization will be further investigated. To achieve good reconstruction under low-count statistics, proper incorporation of prior knowledge, such as tumor distribution from pre-operative MRI, will be studied.

### ADDITIONAL REFERENCES

X. Wu, An efficient anti-aliasing technique, ACM Computer Graphics Siggraph, vol. 25, no. 4, pp. 143-152, 1991.
D. F. Yu and J. A. Fessler, Mean and variance of coincidence photon counting with deadtime, Nucl. Instr. Meth. Phys. Res. A., vol. 488, no. 1-2, pp. 362-374, August 2002.
L. A. Shepp and Y. Vardi, “Maximum likelihood reconstruction for emission tomography” IEEE Trans Med Imaging, vol. MI-1, pp. 113-122, 1982.
H. M. Hudson and R. S. Larkin, “Accelerated Image Reconstruction Using Ordered Subsets of Projection Data”, IEEE Trans Med Imaging, vol. 13, no. 4, December 1994.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A tomographic imaging system for realtime imaging of sub-millimeter-sized tumor clusters in a patient, comprising:
an intra-corporeal component comprising:
i) a first detector in a cavity within the body of a patient, wherein the first detector has an imaging hemisphere;
ii) a radiation source capable of emitting a therapeutic radiation, where said radiation source is also in the cavity within the body of the patient;
a contrast agent, which modulates the intensity of the therapeutic radiation, and
an extra-corporeal component comprising a second detector outside any cavity of the body of the patient, wherein the second detector is configured to always face the imaging hemisphere of said first detector;
wherein the first detector can be rotated with respect to the body of said patient.

2. The system of claim 1, wherein the contrast agent is selected from the group consisting of an iodinated agent, a high-Z material liquid, and a combination thereof.

3. The system of claim 1, wherein the first detector is a gamma detector.

4. The system of claim 1, wherein the second detector is a gamma detector.

5. The system of claim 1, wherein the radiation source is a miniature x-ray source.

6. The system of claim 5, wherein the radiation source further comprises an inlet and an outlet for an x-ray source coolant.

7. The system of claim 1, further comprises:
an additional radiation source for imaging, wherein the additional radiation source is a source of positron-emitters.

8. The system of claim 7, wherein a positron-emitting isotope labeled compound is administered to the patient.

9. The system of claim 1, wherein the radiation source further comprises an anode voltage lead and a cathode voltage lead.

10. The system of claim 1, wherein the radiation source further comprises an inlet and an outlet for the contrast agent.

11. The system of claim 1, wherein the first detector is placed in a cavity selected from the group consisting of the cervical cavity, the ovarian cavity, gloiblastoma multiforme margin cavity, and a surgically created cavity.

12. The system of claim 1, wherein the radiation source can be rotated with respect to the body of said patient.

13. The system of claim 1, wherein the second detector can be rotated with respect to the body of said patient.

14. The system of claim 1, wherein the intra-corporeal component further comprises:
a stationary outer shell that does not rotate with the first detector.

15. The system of claim 1, wherein the intra-corporeal component further comprises:
a shield surrounding the radiation source.

16. The system of claim 15, wherein the shield is removable.

17. A method for imaging a sub-millimeter-sized tumor in a patient, comprising:
administering, to a patient in need, a contrast agent that is preferentially taken up by tumor cells;
placing an intra-corporeal component in a cavity within the body of a patient, wherein intra-corporeal component comprising:
i) a first detector; and ii) a radiation source capable of emitting a therapeutic radiation;
placing a second detector outside any cavity of the body of the patient, wherein the second detector is configured to always face the imaging hemisphere of said first detector;
collecting a first image of a first portion of said cavity using the first detector by using therapeutic x-rays whose intensity is modulated by said contrast agent;
rotating the first and second detector over a first angle while maintaining the relative configuration between them;
collecting a second image of a second portion of said cavity using the first detector, wherein the first and second portions do not overlap completely.

18. The method of claim 17, further comprising:
constructing a new image based on said first and second images.

19. The method of claim 18, wherein the new image comprises three-dimensional information.

20. The method of claim 17, further comprising:
administering a positron-emitting isotope labeled compound to the patient.

21. The method of claim 17, further comprising:
collecting image data from an additional radiation source for imaging, wherein the additional radiation source is a source of positron-emitters.

22. The method of claim 17, wherein the contrast agent is selected from the group consisting an iodinated agent, a high-Z material liquid, and a combination thereof.

23. The method of claim 17, wherein the first angle is between 1 and 180 degrees.

24. The method of claim 23, wherein the first angle is about 180 degrees.
